(19) United States
(12) Patent Application Publication
(10) Pub. No.: US 2009/0281156 A1
(43) Pub. Date: Nov. 12, 2009

(54) ENHANCING PHOTOSTABILIZATION OF OXYMETAZOLINE

(75) Inventors: Nanhye Kim, Germantown, TN (US); Hanwei William Chang, Memphis, TN (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering-Plough Healthcare Products, Inc,

(21) Appl. No.: 12/339,394

(22) Filed: Dec. 19, 2008

Related U.S. Application Data
(60) Provisional application No. 61/015,841, filed on Dec. 21, 2007.

(51) Int. Cl. A61K 31/4164; A61K 31/4402; A61K 31/5415; A61K 31/40; A61K 31/426; A61P 11/02

(52) U.S. Cl. 514/396; 514/357; 514/352; 514/226.2; 514/428; 514/400; 514/370

(57) ABSTRACT

The photostability of oxymetazoline in a topical decongestant composition is enhanced by lowering the pH of the composition using a buffer solution.

This application claims priority from U.S. provisional patent application Ser. No. 61/015,841 filed Dec. 21, 2007.

### FIELD OF THE INVENTION

The field of invention relates to topical decongestants, more specifically, compositions which enhance photostabilization of oxymetazoline and methods of treatment using the same.

### BACKGROUND

Many pharmaceutically active agents are susceptible to photodegradation upon exposure to UV light. Generally, incorporation of light absorbers into formulations can stabilize these photosensitive agents to some extent. For example, N. Jamil et al., “Studies of the photostability of reserpine in parenteral solutions”, Die Pharmazie, 38: 467-469 (1983) refers to studies done on the photostability of reserpine in parenteral formulations and the effects of some commonly used stabilizers. U.S. Pat. No. 6,379,697, titled “Stabilization of photosensitive materials” to Gregoriadis, et al. refers to liposomes containing a photosensitive material together with a light absorbing material capable of increasing the photostability of the photosensitive.

Polyvinylpyrrolidones (PVP) may introduce peroxide impurities into various formulations since its polymerization process involves the use of polymerization initiators such as peroxides, hydroperoxide, and hydrogen peroxides. For example, M. Ashraf-Khorassani et al., “Purification of pharmaceutical excipients with supercritical fluid extraction”, Phar Dev Technol., 4: 507-516 (2005) refers to supercritical fluid extraction's ability to remove common reactive impurities from several pharmaceutical excipients. W. Wasylaschuk et al, “Evaluation of hydroperoxides in common pharmaceutical excipients”, J Pharm Sci., 96: 106-116 (2007) refers to evaluating the hydroperoxide impurity levels of common pharmaceutical excipients.

Upon heat or light exposure, the trace amount of peroxides can decompose into free radicals, which can powerfully catalyze photochemical reactions. Light-induced decomposition of polyoxyethylene chains of polyethylene glycols (PEG) or polysorbate surfactants can also result in the formation of hydrogen peroxides and/or peroxide-free radicals, which lead to fast degradation for the drugs. For example, J. McGinity et al., “Implications of peroxide formation in lotion and ointment dosage forms containing polyethylene glycols”, Drug Dev Commun., 2: 505-519 (1976) refers to studies done on the influence of various factors on the formation rate of peroxide-like impurities in polyethylene glycols. E. Ha et al., “Peroxide formation in Polysorbate 80 and protein stability”, J Pharm Sci., 91: 2252-2264 (2002) refers to studies done on the peroxide formation in Polysorbate 80 under a variety of storage conditions and tested the potential of peroxides in Polysorbate 80 to oxidize a model protein, IL-2 mutein. M. Donbrow, et al., “Autoxidation of polysorbates”, J Pharm Sci., 67: 1676-1681 (1978) refers to aqueous solutions of polysorbate 20 which undergo auto-oxidation on storage.

Numerous methods have been sought to prevent or reduce the photolysis of photosensitive substances in the presence of the above-mentioned excipients. In general, they can be protected from light-induced decomposition by the use of colored containers or a polymer film containing UV absorbers. However, the minimization of the light exposure levels alone in formulations is not always sufficient to prevent apparent photochemical reactions.

Oxymetazoline is a selective alpha-1 agonist and partial alpha-2 agonist topical decongestant, generally available in its salt form (oxymetazoline HCl) in aqueous based formulations. It is used in products such as Neo-Synephrine, Vicks Sinex and Afrin. Oxymetazoline works by constricting blood vessels in your body. For example, oxymetazoline in a nasal formulation acts directly on the blood vessels in your nasal tissues. Constriction of the blood vessels in your nose and sinuses leads to drainage of these areas and a decrease in congestion.

Oxymetazoline hydrochloride has the chemical name 6-tert-butyl-3-(2-imidazolin-2-ylmethyl)-2,4-dimentylphenol hydrochloride (CAS Registry No. 2315-02-8). The molecular weight of oxymetazoline hydrochloride is 296.84 and it has the following chemical structure:

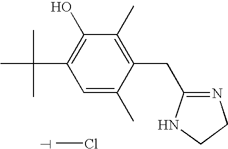

### SUMMARY

Oxymetazoline HCl was found to be light sensitive in aqueous solution and its photodegradation level was substantially increased in the presence of either PVPs or PEGs. Since the concentration of oxymetazoline HCl employed in nasal spray formulations is very low, typically 0.05% w/v, and other components of the formulations (which may be destabilizing) are present in much higher concentrations than the drug itself, there is a particular concern with the decomposition of oxymetazoline HCl.

It has been found that by lowering pH of the formulations containing oxymetazoline HCl, photodegradation level of oxymetazoline HCl is significantly reduced, even in the presence of destabilizing excipients, such as PVPs or PEGs.

One example embodiment of the invention encompasses a topical decongestant composition which includes oxymetazoline HCl; at least one of a polyvinyl pyrrolidone or a polyethylene glycol; and a buffer solution, wherein the composition has a pH of about 3 to about 6. Optionally, the composition has a pH of about 3.5 to about 5.5. Optionally, the composition has a pH of about 4 to about 5. Optionally, the buffering solution includes a buffering agent selected from the group consisting of citric acid, sodium citrate, sodium acetate, acetic acid, dibasic phosphate, monobasic phosphate and combinations thereof. Optionally, the buffering agent is a combination of citric acid and phosphate. Optionally, the buffer solution comprises a citric acid-phosphate solution comprising about 0.1 M citric acid and about 0.2 M monobasic sodium phosphate monohydrate. Optionally, the concentration of oxymetazoline HCl present is from about 0.01% to about 0.10% weight/volume of the composition. Optionally, the concentration of oxymetazoline HCl present is about 0.05% weight/volume of the composition. Optionally, PVP is present from about 0.5% to about 15% by weight/volume, and wherein the PVP has a average molecular weight of about 10,000 to about 40,000. Optionally, PVP is present from about 0.5 to about 3% weight/volume and has an average molecular weight of about 40,000. Optionally, PEG is present from less than about 15% by weight/volume, and wherein the PEG has a average molecular weight of about 400 to about 3350. Optionally, the PEG is present from about 2.5% to about 5% by weight/volume, and wherein the PEG has an average molecular weight of about 1450.

Another example embodiment of the invention encompasses a process for enhancing photostabilization of oxymetazoline HCl in a topical decongestant composition, which includes combining oxymetazoline HCl, at least one of a polyvinyl pyrrolidone or a polyethylene glycol, and a buffer solution into a mixture having a pH of about 3 to about 6. Optionally, the composition has a pH of about 4 to about 5. Optionally, the PVP is included in the mixture from about 0.5% to about 15% by weight/volume, and wherein the PVP has an average molecular weight of about 10,000 to about 40,000. Optionally, the PEG is included in the mixture from less than about 15% by weight/volume, and wherein the PEG has a average molecular weight of about 400 to about 3350. Optionally, the buffer solution comprises one or more buffering agents. Optionally, the buffering agent is selected from the group consisting of citric acid, sodium citrate, sodium acetate, acetic acid, dibasic phosphate, monobasic phosphate and combinations thereof. Optionally, the buffering agent is a combination of citric acid and phosphate. Optionally, the buffer solution is a citric acid-phosphate solution containing about 0.1 M citric acid and about 0.2 M monobasic sodium phosphate monohydrate. Optionally, the concentration of oxymetazoline HCl present is from about 0.01% to about 0.10% weight/volume of the composition.

Another example embodiment of the invention encompasses a method for treating nasal congestion, including administering to a patient a therapeutically effective amount of a topical decongestant composition including oxymetazoline HCl, at least one of a polyvinyl pyrrolidone or a polyethylene glycol, and a buffer solution, wherein the composition has a pH of about 3 to about 6. Optionally, the nasal congestion is a symptom of an affliction selected from the group consisting of allergies, hay fever, sinus irritation or the common cold. Optionally, the topical decongestant composition is selected from the group consisting of a nasal spray, a nasal gel, nose drops and an insufflation. Optionally, the composition is administered to a patient once a day. Optionally, the composition is administered to a patient twice a day. Optionally, the composition is administered to a patient more than twice a day.

Yet another example embodiment of the invention encompasses a nasal administered topical composition including: oxymetazoline HCl; a compound which releases peroxide by decomposition; and a buffer solution, wherein the composition has a pH of about 3 to about 6. Optionally, the composition has a pH of about 3.5 to about 5.5. Optionally, the composition has a pH of about 4 to about 5. Optionally, the buffering solution includes a buffering agent selected from the group consisting of citric acid, sodium citrate, sodium acetate, acetic acid, dibasic phosphate, monobasic phosphate and combinations thereof. Optionally, the buffering agent is a combination of citric acid and phosphate. Optionally, the buffer solution comprises a citric acid-phosphate solution comprising about 0.1 M citric acid and about 0.2 M monobasic sodium phosphate monohydrate. Optionally, the concentration of oxymetazoline HCl present is from about 0.01% to about 0.10% weight/volume of the composition. Optionally, the concentration of oxymetazoline HCl present is about 0.05% weight/volume of the composition. Optionally, the composition further comprising at least one additional pharmaceutically active agent. Optionally, the pharmaceutically active agent is chosen from the group consisting of antihistamines, corticosteroids, and nasal decongestants. Optionally, the antihistamine is chosen from the group consisting of diphenhydramine, chlorpheniramine, tripelennamine, promethazine, clemastine, doxylamine, astemizole, terfenadine, loratadine, desloratadine, cimetidine, famotidine, nizatidine, ranitidine, cromolyn, azatidine, fexofenadine, terfenadine, cetirizine, astemizole, and levocabastine. Optionally, the corticosteroid is chosen from the group consisting of mometasone furoate, dexamethasone, butoxicort, rofleponide, budesonide, deflazacort, ciclesonide, fluticasone, beclomethasone, loteprednol or triamcinolone. Optionally, the nasal decongestant is chosen from the group consisting of levmetamfetamine, ephedrine, ephedrine hydrochloride, ephedrine sulfate, naphazoline hydrochloride, phenylephrine hydrochloride, propylhexedrine, xylometazoline hydrochloride, phenylpropanolamine, phenylephrine and pseudoephedrine.

### DETAILED DESCRIPTION OF EXAMPLE EMBODIMENTS

The amount of oxymetazoline or pharmaceutically acceptable salt thereof found sufficient to effect nasal decongestion is in the range of about 0.01% to about 0.1% by weight/volume of the topical nasal decongestant composition. Typically, 0.05% by weight/volume of oxymetazoline (as the HCl salt) is suitable for adults and children above five years of age. Oxymetazoline HCl is commercially available in products sold by Schering-Plough Corp., Kenilworth, N.J. See also The Merck Index. Tenth Edition, 1983, p. 6838.

Various gums and polymers have been evaluated to determine the suitability of such materials as bioadhesives to extend the nasal muco-cilia clearance time of nasal spray formulations. Desired properties of a bioadhesive include solubility, clarity and compatibility in a conventional nasal spray formulation.

It has been found that polyvinylpyrrolidone (PVP), a linear polymer of 1-vinyl-2-pyrrolidone extends muco-cilia clearance times of nasal spray compositions. Polyvinylpyrrolidones (PVP) is also known as Povidone, and is commercially available as a series of products having mean molecular weights ranging from about 10,000 to about 700,000. The various products are marketed according to average molecular weights designated K-values; e.g., GAF Corporation supplies PVP having K-value=15 as having an average molecular weight of about 10,000, and K-value=30 as having an average molecular weight of about 40,000. The nasal spray compositions of this invention may contain various grades of polyvinylpyrrolidone, i.e., K-15, K-30, K-60 and K-90. The polyvinylpyrrolidone ingredient may be present as one specific grade or as a combination of two or more grades. The most preferable polymer of polyvinylpyrrolidone for the compositions of this invention is Povidone K29-32. Povidone K29-32 having a molecular weight of about 39,450 (sold by General Aniline & Film Corp.) Polyvinylpyrrolidone, when present, is typically present in an amount from about 0.5% to about 15% by weight/volume of the total composition. Preferably, it is present in an amount from about 0.5% to about 3% by weight/volume of the total composition.

Polyethylene glycol (PEG) is a linear polymer formed by the addition reaction of ethylene glycol with ethylene oxide and is commercially available in average molecular weights ranging from about 200 to greater than 20,000. The commercially available grades of polyethylene glycol are marketed based on the average molecular weight, e.g., the grade nomenclature is identified with the molecular weight. For example, PEG 400 represents material with an average molecular weight of 400 and the material with an average molecular weight of 600 is known as PEG 600. PEG 200, 300, 400, and 600 are clear viscous liquids at room temperature; PEG 900, 1000, 1450, 3350, 4500 and 8000 are white, waxy solids. The preferred polyethylene glycols for the compositions of this invention are PEG 400 to PEG 3350; the most preferred polyethylene glycol is PEG 1450. Polyethylene glycol, when present, is typically present in an amount from about 0% to 15% by weight/volume of the total composition. Preferably, it is present in an amount from about 0.5% to 10% by weight/volume of the total composition. More preferably, it is present in an amount from about 2.5% to about 5% by weight/volume of the total composition.

As used herein, the term “McIlvaine buffer” refers to a citric acid-phosphate solution containing about 0.1 M citric acid and about 0.2 M monobasic sodium phosphate monohydrate.

As used herein, the term “Polysorbate 80” (commercially also known as TWEEN® 80, a trademark of Croda International Plc, previously Uniqema/ICI) is a nonionic detergent and emulsifier derived from polyoxylated sorbitol and oleic acid.

### Topical Decongestant Composition

One example embodiment of the present invention encompasses a topical decongestant composition comprising oxymetazoline HCl and a buffer solution, wherein the composition has a pH of about 3 to about 6. The inventors have found a method of enhancing photostabilization of oxymetazoline HCl by lowering the pH of the composition. The appealing aspects of this method are its simple preparation using common buffer solutions, its ability to suppress photochemical reactions, and its potential to offer synergistic effect by combining it with other photo-protection devices.

Typically, the topical decongestant composition has a pH of about 3 to about 6. Preferably, the composition has a pH of about 3.5 to about 5.5. More preferably, the composition has a pH of about 4 to about 5.

The topical decongestant composition can also contain viscosity enhancing agents, typically, the viscosity enhancing agents are polyvinyl pyrrolidones (PVPs) and polyethylene glycols (PEGs). The PEGs may also serve as a moisturizer and PVPs are also used to improve feel in the nose. The PVPs and PEGs may also serve as a bioadhesive, increasing the clearance times of the nasal decongestants.

Typically, the buffer solution contains one or more buffering agents sufficient to adjust and maintain the pH of the compositions from about 3 to about 6. Preferably, the buffering agent is citric acid, sodium citrate, sodium acetate, acetic acid, dibasic phosphate, monobasic phosphate or combinations thereof. More preferably, the buffering agent is a combination of citric acid and phosphate. Most preferably, the buffer solution is a citric acid-phosphate solution comprising about 0.1 M citric acid and about 0.2 M monobasic sodium phosphate monohydrate. Typically, the amount of citric acid present is from about 0.10% to about 0.50% weight/volume of the composition and the amount of monobasic sodium phosphate monohydrate present is from about 0.20% to about 0.65% weight/volume of the composition. Preferably, the amount of citric acid present is 0.20% to 0.45% weight/volume of the composition and the amount of monobasic sodium phosphate present is 0.35% to about 0.60% weight/volume of the composition.

Typically, water is present in the composition, in an amount from about 98% to about 99.5% weight/volume of the composition. Preferably, water is present in an amount from about 99.1% to about 99.2% weight/volume of the composition.

Depending on the intended application, it may be desirable to incorporate up to about 10 percent by weight, more typically about 0.5 to about 5 weight percent, of a rheology-modifying agent, such as a polymer or other material. Useful materials include, without limitation thereto, sodium carboxymethyl cellulose, algin, carageenans, carbomers, galactomannans, hydroxypropyl methylcellulose, hydroxypropyl cellulose, polyethylene glycols, polyvinyl alcohol, polyvinylpyrrolidone, sodium carboxymethyl chitin, sodium carboxymethyl dextran, sodium carboxymethyl starch and xanthan gum. Combinations of any two or more of the foregoing are also useful.

Certain example embodiments of the invention may contain mixtures of microcrystalline cellulose and an alkali metal carboxyalkylcellulose. Such combinations are commercially available, including such examples as Avicel™ RC-591 and Avicel™ RC-581 (FMC Corporation, Philadelphia, Pa. U.S.A.), both of which have the same bulk chemical composition containing approximately 89 weight percent microcrystalline cellulose and approximately 11 weight percent sodium carboxymethylcellulose. Microcrystalline cellulose and alkali metal carboxyalkylcellulose are commercially available separately, and can be mixed in desired proportions for use in the invention, with the amount of microcrystalline cellulose preferably being between about 85 and about 95 weight percent of the mixture for both separately mixed and co-processed mixtures.

These compositions may also contain one or more aromatic alcohols, surfactants, moisturizing agents, antioxidants, stabilizers, antimicrobial preservatives and the like, and mixtures thereof.

Aromatic alcohols may be selected from the group consisting of benzyl alcohol and phenyl ethyl alcohol. The aromatic alcohol, when used, is typically present in an amount from about 0% to about 5% by weight/volume of the total composition. Preferably, it is present in an amount from about 0.2% to about 3% by weight/volume of the total composition, and more preferably, it is present in an amount from about 0.25% to about 1% by weight/volume of the total composition.

Surfactants, such as Polysorbate 80, when used, is typically present in an amount from about 0% to 2.0% by weight/volume of the total composition. Preferably, it is present in an amount from about 0% to 1.5% by weight/volume of the total composition and more preferably, it is present in an amount from about 0% to 1.25% by weight/volume of the total composition.

Moisturizing agents, such as propylene glycol, when used, are typically present in an amount from about 0% to 10% by weight/volume of the total composition. Preferably, it is present in an amount from about 1% to 4% by weight/volume of the total composition and more preferably, it is present in an amount from about 1.5% to 3.5% by weight/volume of the total composition.

Antioxidants, such as disodium EDTA, when used, are typically present in an amount from about 0% to 0.10% by weight/volume of the total composition. Preferably, it is present in an amount from about 0.01% to 0.05% by weight/volume of the total composition and more preferably, it is present in an amount from about 0.015% to 0.03% by weight/volume of the total composition.

Antimicrobial preservative, when used, is typically present in an amount from about 0.01% to about 0.3% by weight/volume of the composition. A typical suitable preservative which functions as an antimicrobial agent includes the commercially available preservative, benzalkonium chloride, in the range of about 0.02 to about 0.025% by weight/volume when present.

Another embodiment of the present invention encompasses a method for treating nasal congestion comprising administering to a patient a therapeutically effective amount of a topical decongestant composition comprising oxymetazoline HCl and a buffer solution, wherein the composition has a pH of about 3 to about 6, which also contains PEGs and/or PVPs, and the buffers described previously. Typically, the nasal congestion is a symptom afflicted from allergies, hay fever, sinus irritation or the common cold. The topical decongestant composition may be in the form of a nasal spray, nasal gel, nose drops or an insufflation.

Typically, the topical decongestant composition is administered to a patient once a day, twice a day or more than twice a day. Prolonged use of these types of sprays can damage the delicate mucous membranes in the nose. As a result, decongestant nasal sprays are advised for short-term use only.

Medicaments of the example embodiments of the present invention may contain additional pharmaceutically active agents in addition to oxymetazoline. Thus, in one aspect of the present invention, oxymetazoline may be combined with a corticosteroid, e.g., mometasone furoate, dexamethasone, butoxicort, rofleponide, budesonide, deflazacort, ciclesonide, fluticasone, beclomethasone, loteprednol or triamcinolone, or combinations thereof.

For the treatment of allergic, non-allergic rhinitis and/or inflammatory diseases of the upper or lower airway passages to treat for example asthma or allergic or non-allergic rhinitis, the substantially non-systematically bioavailable amount of Mometasone Furoate which may be administered as an aqueous suspension or dry powder is in the range of about 10 to 5000 micrograms (“mcg”)/day, 10 to 4000 mcg/day, 10 to 2000 mcg/day, 25-1000 mcg/day, 25 to 400 mcg/day, 25-200 mcg/day, 25-100 mcg/day or 25-50 mcg/day in single or divided doses.

In another aspect of the example embodiment of the present invention, oxymetazoline may be combined with an antihistamine. Antihistamines can be of H₁ or H₂ antagonists or other types of histamine release inhibitors. The H₁ antagonists can be sedating or non-sedating, such as diphenhydramine, chlorpheniramine, tripelennamine, promethazine, clemastine, doxylamine, astemizole, terfenadine, and loratadine, among others. Examples of H₂ antagonists include, but are not limited to, cimetidine, famotidine, nizatidine, and ranitidine. Examples of histamine-release inhibitors include cromolyn. Long-acting antihistamines selected from one or more of the group consisting of loratadine, desloratadine, azatidine, fexofenadine, terfenadine, cetirizine, astemizole, and levocabastine, or their pharmaceutically acceptable salts are suitable for the pharmaceutical compositions of the invention.

Preferred antihistamines include loratadine and desloratadine. Loratadine is disclosed in U.S. Pat. No. 4,282,233 as a non-sedating antihistamine useful, for example, in alleviation of seasonal allergic rhinitis symptoms such as sneezing and itching. The active metabolite of loratadine is desloratadine, which has a half-life (t_{1/2}) of approximately 15 to 19 hours. U.S. Pat. No. 5,595,997 discloses methods and compositions for treating seasonal allergic rhinitis symptoms using desloratadine. Loratadine and desloratadine are available in the form of conventional tablets that release the active agent in a conventional manner. An exemplary formulation releases loratadine by the processes of disintegration and dissolution such that loratadine begins to elicit its antihistaminic effect within 1 to 3 hours and the effect lasts in excess of 24 hours. Due to the long half life of loratadine compared to phenylephrine, the loratadine in the formulation according to the present invention is preferably available for immediate release. For example, loratadine or desloratadine may be present in solution in the carrier liquid of a liquid core or incorporated into the top coating of the product.

Other antihistamines are also useful for the practice of the instant invention. Azatadine is disclosed in Belgian Patent No. 647,043 and in corresponding U.S. Pat. Nos. 3,326,924 and 3,419,565. The elimination half-life is reported to be 9-12 hours. Terfenadine and fexofenadine are disclosed in U.S. Pat. No. 3,878,217 and have a duration of action of 12 to 24 hours, and greater than 24 hours, respectively. Cetirizine is disclosed in U.S. Pat. No. 4,525,358 and is reported to have a duration of action of 12 to 24 hours. Astemizole is disclosed in U.S. Pat. No. 4,219,559 and is reported to have a duration of action greater than 24 hours. Levocabastine is disclosed in U.S. Pat. No. 4,369,184 and is reported to have a duration of action of 16 to 24 hours. The dosage of antihistamine such as loratadine or desloratadine may be present in different concentrations such as 1-20 mg; preferably 2.5 mg, 5 mg, or 10 mg.

Other decongestants may also be used in combination with oxymetazoline in various example embodiments of the present invention. These nasal decongestants may include the sympathomimetic amine nasal decongestants. Those currently approved for topical use in the United States include, without limitation, levmetamfetamine (also known as 1-desoxyephedrine), ephedrine, ephedrine hydrochloride, ephedrine sulfate, naphazoline hydrochloride, phenylephrine hydrochloride, propylhexedrine and xylometazoline hydrochloride. Additional decongestants which may be used include phenylpropanolamine, phenylephrine and pseudoephedrine. Pseudoephedrine as well as pharmaceutically acceptable acid additional salts, e.g., those of HCl or H₂SO₄, is a sympathomimetic drug recognized as a safe therapeutic agent effective for treating nasal congestion and is commonly administered orally and concomitantly with an antihistamine for treatment of nasal congestion associated with allergic rhinitis. The use of pseudoephedrine as a nasal decongestant is preferred in amounts of about 120 mg pseudoephedrine sulfate dosed one to 4 times daily. However, lesser amounts of pseudoephedrine sulfate may be used in combination with oxymetazoline.

Administration may be carried out as set forth herein and as readily apparent to those of ordinary skill in the art.

The compositions containing oxymetazoline with or without one or more additional active agents described herein when formulated for administration using a nebulizer have advantages including but not limited to oral administration, ease of pediatric therapy and/or high dose loading availability. In another example, the compositions containing oxymetazoline with or without one or more of the other active agents described above can be formulated as a metered dose inhaler product that may be administered either orally or nasally simply by switching the actuator that is designed for nasal delivery with an actuator designed for oral delivery.

Having described the invention with reference to certain preferred embodiments, other embodiments will become apparent to one skilled in the art from consideration of the specification. It will be apparent to those skilled in the art that many modifications, both to materials and methods, may be practiced without departing from the scope of the invention.

### EXAMPLES

### Example 1

### Composition

0.05% (w/v) oxymetazoline HCl solutions with pH ranging from 4 to 6 were prepared using McIlvaine buffer systems (Solutions I-III). The compositions for the tested solutions are listed in Table 1. At each pH level, 0.05% oxymetazoline HCl solutions containing either Povidone 29-32 (approximately 3% w/v) or PEG 1450 (approximately 5% w/v) were also made (Solutions IV-VIII).

The composition is prepared in a conventional manner by thoroughly mixing the ingredients at ambient or elevated temperatures in order to achieve solubility of ingredients where appropriate.

TABLE 1
Compositions for Oxymetazoline HCl solutions prepared
using McIlvaine buffers (pH range: 4-6)

| | solution I | solution II | solution III |
|---|---|---|---|
| Ingredient | (% w/v) | (% w/v) | (% w/v) |
| Oxymetazoline HCl USP | 0.05 | 0.05 | 0.05 |
| Water USP purified | 99.13 | 99.16 | 99.18 |
| Monobasic sodium | 0.39 | 0.48 | 0.55 |
| phosphate monohydrate NF | | | |
| Citric acid | 0.43 | 0.31 | 0.22 |
| pH | 4 | 5 | 6 |

### Example 2

Example embodiments were tested for photostability according to methods described in ICH Harmonized Tripartite Guidelines Stability Testing: Photostability Testing of New Drug Substances and Products. Each sample for photostability studies was placed in an enclosed quartz container and exposed to twice the exposure required by ICH photostability guideline (total exposure of 2.4 million lux hours and an integrated near UV energy of 400 watt hours/square meter).

### Results

TABLE 2
Photostability of Oxymetazoline HCl
in McIlvaine buffers (pH range: 4-6)

| | Total Degradation of Oxymetazoline HCl (%) | | |
|---|---|---|---|
| | Sample | | |
| Time | solution I | solution II | solution III |
| (hour) | (pH = 4) | (pH = 5) | (pH = 6) |
| Initial | 0.03 | 0.04 | 0.02 |
| 1 | 0.03 | 0.00 | 0.10 |
| 2 | 0.27 | 0.22 | 0.31 |
| 4 | 0.90 | 0.57 | 0.70 |
| 8 | 1.89 | 1.35 | 1.98 |
| 16 | 3.57 | 2.92 | 4.78 |
| 24 | 4.22 | 5.72 | 9.17 |
| 40 | 9.12 | 13.05 | 71.79 |

TABLE 3
Photostability of Oxymetazoline HCl in McIlvaine buffers (pH range:
4-6) with Povidone K29-32 (approximately 3% w/v) added.

| | Total Degradation of Oxymetazoline HCl (%) | | |
|---|---|---|---|
| | Sample | | |
| Time | solution IV | solution V | solution VI |
| (hour) | (pH = 4) | (pH = 5) | (pH = 6) |
| Initial | 0.00 | 0.00 | 0.00 |
| 1 | 0.19 | 0.80 | 1.13 |
| 2 | 0.53 | 0.77 | 1.85 |
| 4 | 0.73 | 1.11 | 3.22 |
| 8 | 1.23 | 1.36 | 5.40 |
| 16 | 2.52 | 2.67 | 11.85 |
| 24 | 3.96 | 5.44 | 23.80 |
| 40 | 12.34 | 11.65 | 86.56 |

TABLE 4
Photostability of Oxymetazoline HCl in McIlvaine buffers (pH
range: 4-6) with PEG 1450 (approximately 5% w/v) added.

Total Degradation of Oxymetazoline HCl (%)
Sample

| Time | solution VII | solution VIII |
|---|---|---|
| (hour) | (pH = 4) | (pH = 5) |
| Initial | 0.02 | 0.03 |
| 1 | 0.08 | 0.08 |
| 2 | 0.09 | 0.23 |
| 4 | 0.29 | 0.49 |
| 8 | 0.75 | 1.19 |
| 16 | 1.62 | 3.48 |
| 24 | 4.63 | 8.32 |
| 40 | 11.39 | 55.66 |

All of the observed oxymetazoline HCl degradation peaks were calculated and presented in percentage.

The accelerated photostability studies of oxymetazoline HCl in McIlvaine buffer solutions at the pH of typical nasal spray formulations, pH=6, showed that roughly 9% and 72% of the drug was degraded after 24 and 40 hours of UV light exposure, respectively (shown in Table 2). The total % degradation of oxymetazoline HCl in the tested solutions increased with time and pH. Decreasing the pH of the solution from 6 to 4 significantly decreased the level of degradation, demonstrating that lowering the pH is an effective way to stabilize photodegradation of oxymetazoline HCl. The total percent degradation of oxymetazoline HCl in the solutions made at pH 4 (solution I), pH 5 (solution II) and pH 6 (solution III) at T=40 hours are 9.12, 13.05, and 71.79%, respectively.

Subsequently, protection of oxymetazoline HCl in the presence of either Povidone K29-32 or PEG 1450 against photolytic degradation was investigated. The presence of Povidone K29-32 accelerated the rate of photodegradation at pH=6 (about 21% increase in the total photodegradation at T=40 hours), but lowering the pH from 6 to either 5 or 4 significantly reduced the degradation rate (shown in Table 3). As shown in Table 4, incorporating PEG 1450 into solutions resulted in roughly 4-fold increase (T=40 hours) in the total degradation of oxymetazoline HCl even at pH 5. It is readily seen that the degradation of oxymetazoline HCl decreased markedly when the pH decreased from 5 to 4. Thus, the impact of lowering pH on photostabilization of oxymetazoline was also demonstrated in the presence of destabilizing agents, such as PEGs and PVPs. These unexpected experimental findings demonstrate the lower pH on oxymetazoline HCl enhances the photostability effect on oxymetazoline HCl.

What is claimed is:

1. A topical decongestant composition comprising:
oxymetazoline or a pharmaceutically acceptable salt thereof;
at least one of a polyvinyl pyrrolidone or a polyethylene glycol; and
a buffer solution, wherein the composition has a pH of about 3 to about 6.

2. The topical decongestant of claim 1, wherein the composition has a pH of about 3.5 to about 5.5.

3. The topical decongestant of claim 1, wherein the composition has a pH of about 4 to about 5.

4. The topical decongestant of claim 1, wherein the buffering solution includes a buffering agent selected from the group consisting of citric acid, sodium citrate, sodium acetate, acetic acid, dibasic phosphate, monobasic phosphate and combinations thereof.

5. The topical decongestant of claim 4, wherein the buffering agent is a combination of citric acid and phosphate.

6. The topical decongestant of claim 5, wherein the buffer solution comprises a citric acid-phosphate solution comprising about 0.1 M citric acid and about 0.2 M monobasic sodium phosphate monohydrate.

7. The topical decongestant of claim 1, wherein oxymetazoline HCl is present from about 0.01% to about 0.10% weight/volume of the composition.

8. The topical decongestant of claim 7, wherein the concentration of oxymetazoline HCl present is about 0.05% weight/volume of the composition.

9. The topical decongestant of claim 1, wherein PVP is present from about 0.5% to about 15% by weight/volume, and wherein the PVP has a average molecular weight of about 10,000 to about 40,000.

10. The topical decongestant of claim 9, wherein PVP is present from about 0.5 to about 3% weight/volume and has an average molecular weight of about 40,000.

11. The topical decongestant of claim 1, wherein PEG is present from less than about 15% by weight/volume, and wherein the PEG has a average molecular weight of about 400 to about 3350.

12. The topical decongestant of claim 11, wherein the PEG is present from about 2.5% to about 5% by weight/volume, and wherein the PEG has an average molecular weight of about 1450.

13. A process for enhancing photostabilization of oxymetazoline in a topical decongestant composition, comprising: combining oxymetazoline or a pharmaceutically acceptable salt thereof, at least one of a polyvinyl pyrrolidone or a polyethylene glycol, and a buffer solution into a mixture having a pH of about 3 to about 6.

14. The process of claim 13, wherein the composition has a pH of about 4 to about 5.

15. The process of claim 13, wherein the PVP is included in the mixture from about 0.5% to about 15% by weight/volume, and wherein the PVP has an average molecular weight of about 10,000 to about 40,000.

16. The process of claim 13, wherein the PEG is included in the mixture from less than about 15% by weight/volume, and wherein the PEG has a average molecular weight of about 400 to about 3350.

17. The process of claim 13, wherein the buffer solution comprises one or more buffering agents.

18. The process of 17, wherein the buffering agent is selected from the group consisting of citric acid, sodium citrate, sodium acetate, acetic acid, dibasic phosphate, monobasic phosphate and combinations thereof.

19. The process of claim 17, wherein the buffering agent is a combination of citric acid and phosphate.

20. The process of claim 17, wherein the buffer solution is a citric acid-phosphate solution containing about 0.1 M citric acid and about 0.2 M monobasic sodium phosphate monohydrate.

21. The process of claim 13, wherein the concentration of oxymetazoline HCl present is from about 0.01% to about 0.10% weight/volume of the composition.

22. A method for treating nasal congestion, comprising: administering to a patient a therapeutically effective amount of a topical decongestant composition comprising oxymetazoline or a pharmaceutically acceptable salt thereof, at least one of a polyvinyl pyrrolidone or a polyethylene glycol, and a buffer solution, wherein the composition has a pH of about 3 to about 6.

23. The method of claim 22, wherein the nasal congestion is a symptom of an affliction selected from the group consisting of allergies, hay fever, sinus irritation or the common cold.

24. The method of claim 22, wherein the topical decongestant composition is selected from the group consisting of a nasal spray, a nasal gel, nose drops and an insufflation.

25. The method of claim 22, wherein the composition is administered to a patient once a day.

26. The method of claim 22, wherein the composition is administered to a patient twice a day.

27. The method of claim 22, wherein the composition is administered to a patient more than twice a day.

28. A nasal administered topical composition, comprising:
oxymetazoline or a pharmaceutically acceptable salt thereof;
a compound which releases peroxide by decomposition; and
a buffer solution, wherein the composition has a pH of about 3 to about 6.

29. The composition of claim 28, wherein the composition has a pH of about 3.5 to about 5.5.

30. The composition of claim 28, wherein the composition has a pH of about 4 to about 5.

31. The composition of claim 28, wherein the buffering solution includes a buffering agent selected from the group consisting of citric acid, sodium citrate, sodium acetate, acetic acid, dibasic phosphate, monobasic phosphate and combinations thereof.

32. The topical decongestant of claim 28, wherein the buffering agent is a combination of citric acid and phosphate.

33. The composition of claim 28, wherein the buffer solution comprises a citric acid-phosphate solution comprising about 0.1 M citric acid and about 0.2 M monobasic sodium phosphate monohydrate.

34. The composition of claim 28, wherein oxymetazoline HCl is present from about 0.01% to about 0.10% weight/volume of the composition.

35. The composition of claim 34, wherein the concentration of oxymetazoline HCl present is about 0.05% weight/volume of the composition.

36. The composition of claims 1 or 28 further comprising at least one additional pharmaceutically active agent.

37. The composition of claim 36 wherein the pharmaceutically active agent is chosen from the group consisting of antihistamines, corticosteroids, and nasal decongestants.

38. The composition of claim 37, wherein the antihistamine is chosen from the group consisting of diphenhydramine, chlorpheniramine, tripelennamine, promethazine, clemastine, doxylamine, astemizole, terfenadine, loratadine, desloratadine, cimetidine, famotidine, nizatidine, ranitidine, cromolyn, azatidine, fexofenadine, terfenadine, cetirizine, astemizole, and levocabastine.

39. The composition of claim 37, wherein the corticosteroid is chosen from the group consisting of mometasone furoate, dexamethasone, butoxicort, rofleponide, budesonide, deflazacort, ciclesonide, fluticasone, beclomethasone, loteprednol or triamcinolone.

40. The composition of claim 37, wherein the nasal decongestant is chosen from the group consisting of levmetamfetamine, ephedrine, ephedrine hydrochloride, ephedrine sulfate, naphazoline hydrochloride, phenylephrine hydrochloride, propylhexedrine, xylometazoline hydrochloride, phenylpropanolamine, phenylephrine and pseudoephedrine.
